(19) United States
(12) Patent Application Publication
(10) Pub. No.: US 2014/0275832 A1
(43) Pub. Date: Sep. 18, 2014

(54) DEVICE AND METHOD FOR OBTAINING VITAL SIGN INFORMATION OF A SUBJECT

(71) Applicant: KONINKLIJKE PHILIPS N.V., EINDHOVEN (NL)

(72) Inventors: Jens MUEHLSTEFF, Aachen (DE); Marek Janusz BARTULA, Eindhoven (NL); Erik BRESCH, Eindhoven (NL); Siegfried Walter KAESTLE, Nuffringen (DE); Caifeng SHAN, Eindhoven (NL)

(73) Assignee: KONINKLIJKE PHILIPS N.V., EINDHOVEN (NL)

(21) Appl. No.: 14/200,556

(22) Filed: Mar. 7, 2014

Related U.S. Application Data
(60) Provisional application No. 61/781,134, filed on Mar. 14, 2013.
(60) Provisional application No. 61/834,909, filed on Jun. 14, 2013.

(30) Foreign Application Priority Data
Mar. 14, 2013 (EP) 13159124.0
Jun. 14, 2013 (EP) 13172132.6

(51) Int. Cl. A61B 5/00; A61B 5/0402; A61B 5/0205

(52) U.S. Cl. CPC A61B 5/6887; A61B 5/0205; A61B 5/0402; A61B 5/7207
USPC 600/301

(57) ABSTRACT

For the measurement of vital sign information such as a respiratory rate and a heart rate a device for obtaining vital sign information of a subject is provided, comprising a first detection unit that acquires first set of detection data allowing the extraction of a first vital sign information signal related to a first vital sign of the subject and a second detection unit that acquires a second set of detection data allowing the extraction of a second vital sign information signal related to a second vital sign of the subject. An analysis unit extracts the first vital sign information signal from the first set of detection data (3a) and extracts the second vital sign information signal from the second set of detection data. A processing unit combines the first vital sign information signal and the second vital sign information signal to obtain a combined vital sign information signal. An extracting unit extracts at least one of the first and second vital signs of the subject from the combined vital sign information signal.

### CROSS REFERENCE TO RELATED APPLICATIONS

This application claims the benefit of U.S. provisional application Ser. No. 61/781,134 filed Mar. 14, 2013 and U.S. provisional application Ser. No. 61/834,909 filed Jun. 14, 2013 and European provisional application serial no. 13159124.0 filed Mar. 14, 2013 and European provisional application serial no. 13172132.6 filed Jun. 14, 2013, all of which are incorporated herein by reference.

### FIELD OF THE INVENTION

The present invention relates to a device, method, processing apparatus, processing method and computer program for obtaining vital sign information, in particular the respiratory rate and/or the heart rate, of a subject.

### BACKGROUND OF THE INVENTION

Vital signs of a person, for example the heart rate (HR) or respiratory information (respiratory parameters) such as the respiratory rate (RR), can serve as a powerful predictor of serious medical events. For this reason the respiratory rate and/or the heart rate are often monitored online in intensive care units or in daily spot checks in the general ward of a hospital. Besides the heart rate, the respiratory rate is one of the most important vital signs. Both, the HR and the RR are still difficult to measure without having direct body contact. In present intensive care units, thorax impedance plethysmography or the respiratory inductive plethysmography are still the methods of choice for measuring the RR, wherein typically two breathing bands are used in order to distinguish thorax and abdominal breathing motion of a person. The HR is typically measured by use of electrodes, fixed at the chest of the subject, wherein the electrodes are connected to remote devices through cables. However, these obtrusive methods are uncomfortable and unpleasant for the patient being observed.

Moreover, unobtrusive respiratory rate measurements can be accomplished optically by use of a stationary video camera. A video camera captures the breathing movements of a patient's chest in a stream of images. The breathing movements lead to a temporal modulation of certain image features, wherein the frequency of the modulation corresponds to the respiratory rate of the patient monitored. Examples of such image features are the average amplitude in a spatial region of interest located around the patient's chest, or the location of the maximum of the spatial cross correlation of the region of interest in subsequent images.

Further, one or more video cameras are used for unobtrusively monitoring the HR, the RR or other vital signs of a subject by use of remote photoplethysmographic imaging. Remote photoplethysmographic imaging is, for instance, described in Wim Verkruysse, Lars O. Svaasand, and J. Stuart Nelson, “Remote plethysmographic imaging using ambient light”, Optics Express, Vol. 16, No. 26, December 2008. It is based on the principle that temporal variations in blood volume in the skin lead to variations in light absorptions by the skin. Such variations can be registered by a video camera that takes images of a skin area, e.g. the face, while the pixel average over a selected region (typically part of the cheek in this system) is calculated. By looking at periodic variations of this average signal, the heart rate and respiratory rate can be extracted. There are meanwhile a number of further publications and patent applications that describe details of devices and methods for obtaining vital signs of a patient by use of remote PPG.

Thus, the pulsation of arterial blood causes changes in light absorption. Those changes observed with a photodetector (or an array of photodetectors) form a PPG (photo-plethysmography) signal (also called, among other, a pleth wave). Pulsation of the blood is caused by the beating heart, i.e. peaks in the PPG signal correspond to the individual beats of the heart. Therefore, a PPG signal is a heart rate signal in itself. The normalized amplitude of this signal is different for different wavelengths, and for some wavelengths it is also a function of blood oxygenation or other substances found in blood or tissue.

Moreover, unobtrusive non-camera based systems for obtaining vital sign information are also known. These systems are based on a surface structure comprising sensor units, which are in unobtrusive contact with the subject for obtaining vital sign information of the subject. Such systems are typically embodied in mattresses or textile structures, being in close proximity to the subject. The sensor units typically comprise pressure sensors for measuring pressure or weight distribution or time-dependent changes thereof and/or inductive sensors for measuring vital sign information, in particular ECG signals related to the heart rate.

The quality and the reliability of the vital sign information obtained by a camera based system are largely influenced by the quality of the input image data influenced by an appropriate selection of the image contrast and the selected region of interest.

Further, the obtained image data, such as a stream of captured images representing radiation reflected or emitted from the subject, generally comprise, besides the desired signal to be extracted, further signal components from overall disturbances, such as noise due to changing luminescence conditions or disturbing motions of observed objects.

Moreover, the quality and the reliability of the vital sign information obtained by the non-camera based systems are also affected by overall disturbances, such as movement of the subject and/or contact problems of the sensors used.

With respect to camera based systems, even a superposition of vital sign signals, such as a respiratory rate signal superimposed by a heart rate signal or vice versa, adversely affect the determination of respiratory information.

Such a superposition of vital signs can e.g. be measured, when a camera system observes the thorax motion of a subject, wherein the thorax motion due to breathing is superimposed by movements related to heart rate signals, so-called cardiac seismograms. These superimposed signals can have a comparable magnitude and even a comparable frequency. This might lead to dangerous situations, in particular during a period without breathing. Errors can occur that are related to the superimposed heart rate signal, which could give the impression that a respiratory rate is detected, in which an apnea phase is present.

WO 2012/140531 A1 discloses a respiratory motion detection apparatus for detecting the respiratory motion of a person. This detection apparatus detects electromagnetic radiation emitted and/or reflected of a person wherein this electromagnetic radiation comprises a continuous or discrete characteristic motion signal related to the respiratory rate of the person and other motion artifacts related to the movement of the person or related to ambient conditions. This apparatus increases the reliability of the respiratory rate measurement by taking into account data processing means adapted to separate the respiratory rate signal from overall disturbances by taking into account a predefined frequency band, common predefined direction or an expected amplitude band and/or amplitude profile to distinguish the different signals.

### SUMMARY OF THE INVENTION

It is therefore an object of the invention to provide a device and a method as well as a processing apparatus and processing method for reliably obtaining vital sign information of a subject.

It is further an object of the invention to provide a device and a method as well as a processing apparatus and processing method for reliably obtaining respiratory information of a subject, in particular the respiratory rate of a subject.

It is further an object of the invention to provide a device and a method for extracting respiratory information of the subject from detected motion signals providing further refinements facilitating obtaining the desired signals with little efforts, especially reduced calculation and computing requirements.

In a general aspect of the present invention a device for obtaining vital sign information of a subject is presented that comprises a first detection unit that acquires a first set of detection data allowing the extraction of a first vital sign information signal related to a first vital sign of the subject, a second detection unit that acquires a second set of detection data allowing the extraction of a second vital sign information signal related to a second vital sign of the subject, an analysis unit that extracts the first vital sign information signal from the first set of detection data and that extracts the second vital sign information signal from the second set of detection data, a processing unit that combines the first vital sign information signal and the second vital sign information signal to obtain a combined vital sign information signal, and an extracting unit that extracts at least one of the first and second vital signs of the subject from the combined vital sign information signal.

In an embodiment according to the general aspect of the present invention, a processing apparatus that obtains vital sign information of a subject is presented that comprises an analysis unit that extracts a first vital sign information signal from a first set of detection data and that extracts a second vital sign information signal from a second set of detection data, a processing unit that combines the first vital sign information signal and the second vital sign information signal to obtain a combined vital sign information signal, and an extracting unit that extracts at least one of the first and second vital signs of the subject from the combined vital sign information signal.

In another embodiment of the general aspect of the present invention, there is provided a computer program which comprises program code means for causing a computer to perform the steps of one of the methods disclosed herein when said computer program as carried out on a computer. Further, a non-transitory computer-readable recording medium that stores therein such a computer program product, which, when executed by a processor, causes said steps of the method disclosed herein to be performed, is presented.

Prior art detection devices can be grouped into unobtrusive devices and obtrusive devices. The unobtrusive devices typically comprise camera systems and/or sensor systems comprising unobtrusive sensors, such as capacitive sensor and/or pressure sensors for obtaining detection data from the subject observed, where the vital sign information, especially the heart rate information and/or the respiratory information, is extracted from. The inventors have found that the known unobtrusive devices partially fail to produce reliable vital sign measurements. The quality of vital signs measured by such unobtrusive devices are typically dependent on error signals related to movements of the subject, poor ambient conditions or contact problems of the sensors used, which could lead to a misinterpretation of the obtained signals.

For this reason the processing unit is configured to combine the first vital sign information signal and the second vital sign information signal to obtain a combined vital sign information signal in way, that the combined vital sign information signal is less affected by error signals. Based on this combined vital sign information signal the extracting unit extracts at least one of the first and second vital sign information signals of the subject.

Preferred embodiments of the invention are defined in the dependent claims. It shall be understood that the claimed processing apparatus, methods and computer program have similar and/or identical preferred embodiments as the claimed device and as defined in the dependent claims.

The term “vital sign” as used herein refers to a physiological parameter of a subject. In particular, the term “vital sign” comprises the heart rate (HR) and the respiratory rate (RR). The term “parameter” or “information” as used herein refers to quantity being extracted from measured signals related to the respective “vital signs”.

In a first aspect of the invention the device for obtaining vital sign information of the subject is a device for obtaining respiratory information of the subject that comprises an imaging unit for acquiring a first set of image data detected from a skin portion of the subject allowing the extraction of a heart rate signal related to a heart rate of a subject and a second set of image data detected from a body portion of the subject allowing the extraction of a motion signal related to respiratory information of the subject, an analysis unit for extracting the heart rate signal from the first set of image data and for extracting the motion signal from the second set of image data, wherein the motion signal comprises a superposition of respiratory information and heart rate information, a processing unit for at least partially removing the heart rate information from the motion signal by use of the extracted heart rate signal, and an extracting unit for extracting respiratory information of the subject from the processed motion signal.

In an embodiment of the first aspect of the present invention, a processing apparatus for obtaining respiratory information of a subject is presented that comprises an analysis unit for extracting a heart rate signal from a first set of image data detected from a skin portion of the subject allowing the extraction of a heart rate signal related to a heart rate of the subject and for extracting the motion signal from the second set of image data detected from a body portion of the subject allowing the extraction of a motion signal related to respiratory information of the subject, wherein the motion signal comprises a superposition of respiratory information and heart rate information, a processing unit for at least partially removing the heart rate information from the motion signal by use of the extracted heart rate signal, and an extracting unit for extracting respiratory information of the subject from the processed motion signal.

Prior art detection devices can be grouped into unobtrusive devices and obtrusive devices. The unobtrusive devices typically comprise a camera for obtaining images or a stream of images, wherein the vital sign information, especially the heart rate information and/or the respiratory information is extracted from. The obtrusive devices comprise detectors that are in direct contact with the body of the subject, e.g. a patient.

The inventors have found that especially the unobtrusive devices according to prior art partially fail to produce reliable vital sign measurements. The quality of the vital signs measured by the unobtrusive devices are typically dependent on error signals related to the movement of the patient, which could lead to a misinterpretation of the obtained signals. However, it is already well-known in the art that such error signals can well be separated or removed, since vital sign signals and the error signals usually have different characteristics. A different problem arises, when the desired vital sign is related to a signal being superimposed by a different vital sign signal having a similar characteristic, for instance similar amplitude and/or similar frequency. The inventors have found that these kinds of signals can be misinterpreted as a pathological condition or a critical event of the monitored subject can be missed.

Therefore, especially the camera based unobtrusive monitoring devices fail by extracting the respiratory rate that is related to an at least partially periodic motion signal, wherein the motion signal is superimposed by a heart rate signal being periodic as well.

According to the first aspect of the present invention, the device comprises an imaging unit for acquiring a first set of image data being detected from a skin portion of the subject. The imaging unit represents the first detection unit and the second detection unit.

The skin portion is typically a region of the body having a good blood circulation. Based on these first image data, a heart rate signal can be extracted, related to the heart rate of the subject observed, in particular using a method well-known in the art in the field of remote photo-plethysmography (PPG). These known methods can comprise the analysis of subtle color changes of the skin regions of the person, wherein these subtle color changes are related to the heart rate or different heart-related signals inter alia the oxygen saturation of the blood. Such methods are known in the art and are commonly used for e.g. extracting heart rate information of a person from PPG signals.

The imaging unit is further adapted to acquire a second set of image data, being detected from a body portion of the subject allowing the extraction of a motion signal related to the respiratory rate information of the subject. By way of example, a body portion is typically the chest of the person or the nose or even other areas of the body of the subject, where respiratory motion can be detected.

The analysis unit according to the first aspect of the present invention is adapted to extract the heart rate signal from the first set of image data, wherein the above mentioned known analysis methods can be used. In addition, the analysis unit is also adapted to extract the motion signal from the second set of image data, wherein the motion signal comprises a superposition of respiratory information and heart rate information. It is to be understood that the motion signal can additionally comprise further error signals related to movement of the subject or related to obtrusive ambient conditions, wherein these error signals can be reduced by known methods, such as the method described in WO 2012/140531 A1, which description is herein incorporated by reference. These methods can e.g. further comprise Fourier filters that are adapted to separate at least partially periodic signals e.g. related to the respiratory rate and/or the heart rate from non-periodic signals. The non-periodic signals are typically related to error signals due to ambient conditions or the movement of the subject. Also filter windows, particularly adapted to a predefined amplitude, a cut of time and/or a cut-off frequency can be taken into account.

The processing unit according to the first aspect of the present invention is adapted for at least partially removing the heart rate information from the motion signal by use of the extracted heart rate signal. The fact that an independent heart rate signal is extracted from the first set of image data is advantageous, since this signal can be used for triggering the removing of the heart rate information that is superimposed upon the motion signal. Therefore, the characteristics of the heart rate signal extracted from the first set of image data, especially the frequency, the amplitude and the signal shape, can be used to determine the heart rate information that is superimposed upon the motion signal and to subsequently remove heart rate information from the motion signal.

Removing the superimposed heart rate information from the motion signal is an important measure of the invention, since during apnea phases, when no motion related to respiration of the subject occurs, the motion signal related to the heart rate can be misinterpreted as a respiratory movement. This might be dangerous, in particular by monitoring little children, since little children often have higher breathing frequencies being comparable to heart rate frequencies. Moreover, undetected apnea phases hidden by a heart rate signal might lead to dangerous situations in general.

The extracting unit according to the first aspect of the present invention is adapted to extract respiratory information of the subject from the processed motion signal. The extraction of respiratory information is therefore the final step to obtain respiratory information that is reliably related to the respiratory rate of the monitored subject.

In a further embodiment of the present invention, said imaging unit comprises a single camera for detecting electromagnetic radiation at least in the visible and/or infrared spectral range. When observing the subject, vital sign signals, especially the heart rate signal and the signal related to respiratory information, can be derived from slight variations in the radiation emitted, e.g. infrared light, and/or reflected e.g. visible light. For instance, the use of a camera that is sensitive especially in the infrared spectral range can be advantageous, since regions having body temperature can well be separated from ambient objects. For everyday applications it could be appreciated if mainly visible light is detected and analysed. For an application during a sleep period of the person, wherein the ambient light conditions are quite poor, it could also be advantageous to detect infrared light emitted or reflected from the person. To this end, besides common natural or artificial light sources no further radiation sources are required and/or have to be considered during analysis.

This embodiment can be further developed in that the camera is adapted for capturing a signal within a signal space selected from the group consisting of RGB, sRGB, Rg chromaticity, HSV, HSL, CHYK, YPbPr, YCbCr, xvYCC, and combinations thereof. It goes without saying that also normalizing measures can be applied to the first and second set of image data so as to obtain signals being less affected by varying illumination conditions.

In other words, cameras that are able to record single images or series of single images, especially video cameras providing a sufficient color depth (even so-called webcams and/or cameras in mobile devices), can be utilized for observing the subject of interest and acquiring (recording) the first and second set of image data to be analysed. Further, also derivatives of the mentioned signal space types may be utilized, such as logRGB. It can be further envisaged to combine several distinct signal spaces at least partially so as to provide a broader spectral basis for the required analysing processes.

According to another embodiment of the present invention the imaging unit is configured to simultaneously acquire the first set of image data and the second set of image data. Simultaneously monitoring both, the first set of image data allowing the extraction of the heart rate signal and the second set of image data allowing the extraction of the motion signal related to respiratory information of the subject, is advantageous since only one camera is required. The camera comprises a field of view covering both, the skin portion of the subject and the body portion of the subject. By way of example, the field of view of the camera can be adjusted such that the chest of the subject and the face of the subject are covered by the field of view of the camera. The image is typically acquired by an image sensor comprising a plurality of pixels arranged in a two-dimensional matrix. To clearly distinguish the skin portion from the body portion, spatially separated detection windows can be defined to clearly distinguish both portions from one another.

According to another embodiment of the invention the processing unit comprises a notch filter triggered by the heart rate signal and allowing for at least partially removing the heart rate information from the motion signal. A notch filter is a simple and efficient element to suppress and/or remove not desired signals and/or signal components from a number of superimposed signals, wherein the desired signals can easily be extracted afterwards.

According to an advantageous embodiment, the second set of image data is first transformed from time domain to frequency domain, for example by discrete and/or continuous Fourier transformation, and the Fourier spectrum is analysed afterwards. The heart rate extracted from the first set of image data can be used to trigger on the heart rate information, which is a superimposed portion of the motion signal. Processing the motion signal in the frequency domain is advantageous since the signal portion related to the heart rate can simply be cut out by the notch filter triggered by the heart rate signal. It is to be understood that also derivatives of the heart rate signal, e.g. the amplitude and/or the heart rate signal shape, can be used for triggering. In a subsequent step, the cleaned signal can be back transferred back from frequency to time domain, while only the motion signal related to respiratory information remains.

According to another advantageous embodiment the notch filter is adapted for cutting out portions of the motion signal in the time domain, which are related to the heart rate signal of the first set of image data.

With respect to the latter two embodiments it is to be noted that the notch filter needs not necessarily be triggered to the heart rate derived from the heart rate signal only, but can also be triggered to other derivatives of the heart signal, especially the amplitude or the signal shape of the heart rate signal.

In a further advantageous embodiment the notch filter comprises filter windows that are dynamically adaptable to the heart rate signal. It is important to have the ability to adapt the filter window of the notch filter to the heart rate signal since the heart rate frequency and the amplitude are typically not constant over time. The heart rate can e.g. be influenced by the physical activity of the subject and/or by disease. Therefore, the size of the filter window in both, frequency and/or time domain, can therefore be well-adapted to a varying heart rate.

According to another embodiment of the present invention the heart rate signal comprises superimposed further respiratory information, wherein the analysis unit is configured to extract further respiratory information by use of the heart rate signal. The heart rate signal detected by the imaging unit is a signal where a repeating smooth, double-humped, cardiac pulse waveform sits on top of a large constant baseline component, which is called the DC component. This modulation is called DC baseline modulation and is related to venous return secondary to changes in intrathoracic pressure throughout the respiratory cycle, which cause a baseline DC modulation of the heart rate signal. During inspiration, decreases in intrathoracic pressure result in a small decrease in central venous pressure increasing venous return. The opposite occurs during expiration. As more blood is shunted from the low pressure venous system at the probe site and the venous bed cyclically fills and drains, the baseline is modulated accordingly. Another effect which modifies the pulse shape of the heart rate signal is the pulse amplitude modulation (PAM). This effect is based on a decreased left ventricular stroke volume, due to changes in intrathoracic pressure during inspiration, which leads to decreased pulse amplitude during this phase of respiration. A further effect which influences both, the pulse shape and rate is the respiratory sinus arrhythmia (RSA). This effect is related to a variation in heart rate that occurs throughout the respiratory cycle. For instance, the effect of RSA is influenced by several factors including age, disease status and physical fitness. Therefore, these three main respiratory modulations that are superimposed on the heart rate signal may be present in varying degrees across the subject's population. In fact, for some subjects only one modulation type may be clearly observed. Therefore, at least one of these modulations can be taken into account to extract the further respiratory information from the heart rate signal.

According to an advantageous embodiment the analysis unit is adapted to perform continuous wavelet transformations of the heart rate signal allowing for extracting further respiratory information from the heart rate signal. The generally known continuous wavelet transformation is a simple and efficient method to extract respiratory information, in particular the respiratory rate, from at least one of the three modulations—DC baseline, PAM and RSA-superimposed on the heart rate signal. As an alternative, further known methods suitable to extract respiratory information from modulated heart rate signals can additionally or as an alternative be taken into account, e.g. comprising short-time Fourier Analysis (STFT), neural networks, and/or variable frequency complex demodulation methods (VFCDM).

According to another embodiment of the present invention the device further comprises a comparing unit for comparing respiratory information extracted from the motion signal and further respiratory information extracted from the heart rate signal. By use of the comparing unit a validity crosscheck can be easily performed if the respiratory information extracted from the motion signal leads to proper results. It is to be understood that the respiratory information extracted from the motion signal can be crosschecked by further respiratory information extracted from the heart rate signal and vice versa.

According to another embodiment of the present invention the device further comprises a user interface that enters information allowing for selecting and/or predefining the skin portion and/or the body portion. It is to be understood that the user can on the one hand manually predefine the field of view of the detection unit such, that the chest and the face of the person are enclosed within the field of view. This is a rough estimation, since the parameters related to the subject can vary over time, by way of example, when the person moves or the person is at least partially covered with cloth or a blanket. On the other hand the field of view of the camera can be adapted automatically to have an optimized aspect ratio between the portions that shall be observed and the background. For this purpose, the subject typically carries one or more markers or orientation indicators detectable by the camera allowing for adapting the field of view in an optimized manner. By way of example, in a hospital the marker or the orientation indicator is typically attached to the body, especially at the skin portion or at the body portion of a patient, which are to be observed. Therefore, the device can be adapted to find these markers, and adapt the field of view in a way that both, the skin portion and the body portion, are well-positioned within the field of view. On the other hand a proper motion signal for extracting the respiratory rate and a proper heart signal for extracting the heart rate of the subject and/or further respiratory information can be detected.

According to a further advantageous embodiment the device further comprises an initializing unit for selecting and/or predefining the skin portion and the body portion based on entered information and/or information related to the subject. The initializing unit is adapted for selecting and/or predefining the skin portion and the body portion, e.g. based on the markers and/or based on orientation indicators attached to the subject. This is a simple way to obtain proper results from the extracted respiratory information since the initializing unit gives feedback to the device, in particular the camera, to follow the predefined skin and body portions automatically based on the given information. A movement of the subject out of the field of view can therefore be avoided.

In a second aspect of the present invention the first detection unit comprises an imaging unit for acquiring a set of image data representing the first set of detection data allowing the extraction of the first vital sign information signal related to the first vital sign of the subject, wherein the second detection unit comprises a sensor unit for acquiring a second set of sensor data detected from a body portion of the subject representing the second set of detection data allowing the extraction of the second vital sign information signal related to the first vital sign of the subject, wherein the first vital sign and the second vital sign are identical, wherein the processing unit is configured to weight the first vital sign information signal by use of a first quality index, to weight the second vital sign information signal by use of a second quality index and to combine the weighted first vital sign information signal and the weighted second vital sign information signal to obtain the weighted vital sign information signal.

As already known, prior art detection devices can be grouped into unobtrusive devices and obtrusive devices. As found by the inventors, the unobtrusive devices according to prior art typically fail to produce reliable vital sign measurements, since the quality of the vital signs measured by the unobtrusive devices are typically dependent on error signals related to movement of the subject or other error signals, which are dependent on contact problems or other ambient insufficiencies, such as a poor illumination level.

These imaging units and/or sensor units comprise sensors and are in unobtrusive contact to the subject, wherein these imaging units and/or sensor units comprise specific disadvantages and advantages with respect to each other. Aiming at an extraction of one specific vital sign, the use of data, which are completely independent from each other, as is the case when using data obtained by the imaging unit and the sensor unit, is advantageous, since at least some of the disadvantages of one of these units are typically not present at the other unit at the same time. Therefore, the first set of data obtained by the imaging unit and the second set of data obtained by the sensor unit are weighted by a first and a second quality index, wherein the first quality index and the second quality index are dependent on the reliability of the data sets obtained. Therefore, a weighted vital sign information signal can be obtained, where the specific insufficiencies of the specific data sets obtained from the imaging unit and/or the sensor unit are considered.

According to a further advantageous embodiment the processing unit is configured to derive the first quality index from the set of image data and the second quality index from the set of sensor data. Taking into account the set of image data and the set of sensor data is advantageous, since these data are directly related to the imaging unit and the sensor unit and their specific insufficiencies. The specific quality indices can be directly derived therefrom, wherein the specific insufficiencies of the imaging unit and the sensor unit are considered.

It is to be understood that typical insufficiencies of the data sets are related to measurement artifacts, caused by the imaging unit and/or the sensor unit and/or artifacts caused by the subject itself and/or caused by poor ambient conditions, such as a poor illumination level.

According to a further advantageous embodiment, the processing unit is configured to derive the first quality index from one or more features of the set of image data and/or environmental data of the environment of the subject, in particular from one or more of an illumination parameter of the subject's illumination, an amplitude, shape and/or variability shape of the first vital sign information signal and motion artifacts, and to derive the second quality index from the set of sensor data for one or more features of the sensor data and/or environmental data of the environment of the subject. Taking into account the features extracted from the set of image data and/or the set of sensor data is advantageous, since no further data sets for deriving the quality indices therefrom are necessary. By way of example, having a poor illumination level, a vital sign information signal extracted from the set of image data is weighted with a low quality index compared to the same vital sign information signal extracted from the set of sensor data, since the vital sign extracted from the set of image data is deemed to have a poor signal quality, which is caused by the poor illumination level, wherein the set of sensor data measured, is not affected by the illumination level at all.

According to a further advantageous embodiment of the device, the sensor unit comprises one or more capacitive sensors for acquiring ECG information of the subject and/or pressure sensors that acquires weight information of the subject. The use of capacitive sensors and/or pressure sensors is advantageous, since these types of sensors are unobtrusive during measurement. These sensors can be integrated into mattresses or textile structures, which are worn by the subject.

The quality index is a factor between 0 and 1, wherein the specific value used is dependent on certain reference values related to the vital sign information signals weighted. The reference value can also be dependent on the background illumination level especially for vital sign information signals being extracted from the image data. The quality index is used as a factor, which is multiplied to the specific vital sign information signal, from which the vital sign is aimed to be extracted. A combined vital sign information signal based on specific vital sign information signals extracted from the imaging data and the sensor data can be obtained by using different weighting schemes or fuzzy logic, which are used to properly combine the specific vital sign information signals.

In a third aspect of the present invention the proposed comprises an imaging unit, representing the first detection unit and the second detection unit, for acquiring a first set of image data detected from a skin portion of the subject allowing the extraction of a first respiratory signal related to a respiratory information of the subject and a second set of image data detected from a body portion of the subject allowing the extraction of a second respiratory signal related to respiratory information of the subject. Further, the proposed device comprises a processing unit that is configured to weight the first respiratory signal by use of a first quality index, to weight the second respiratory signal by use of a second quality index and to combine the weighted respiratory signal and the weighted motion signal to obtain a weighted combined respiratory signal. In this way a more accurate and reliable respiratory information can be derived. In embodiments it is possible to use two or more cameras as imaging units and/or some or more sensors for additionally obtaining respiratory information which may also be combined with the respiratory signals to obtain the weighted combined respiratory signal.

It is to be noted that generally a quality index may also have a value of 0 or 1, i.e. a signal may be completely included (alone) or completely excluded from a weighted combination.

### BRIEF DESCRIPTION OF THE DRAWINGS

These and other aspects of the invention will be apparent from and elucidated with reference to the embodiments described hereinafter. In the following drawings

FIG. 1a shows an exemplary embodiment of a device for obtaining vital sign information of a subject,

FIG. 1b shows a further embodiment of the device for obtaining respiratory information of the subject according to the present invention;

FIG. 2a shows an example graph of the heart rate signal extracted from a first set of image data detected from a skin portion;

FIG. 2b shows a further example graph of a motion signal extracted from a second set of image data detected from a body portion;

FIG. 2c shows a motion signal and a cleaned motion signal in detail;

FIG. 3a shows yet another example graph of a heart rate signal modulated by a respiratory signal;

FIG. 3b shows a first set of a cleaned heart rate signal and a second set of cleaned respiratory signals;

FIG. 4 shows a second embodiment of the device for obtaining respiratory information of the subject according to the present invention;

FIG. 5 shows a further embodiment of the device for obtaining respiratory information of the subject according to the present invention;

FIG. 6 shows a further embodiment of the device for obtaining vital sign information of the subject according to the present invention;

FIG. 7 shows a process flow for obtaining vital sign information of the subject; and

FIG. 8 shows a further embodiment of the device for obtaining respiratory information of the subject according to the present invention.

### DETAILED DESCRIPTION OF THE INVENTION

FIG. 1a shows a first exemplary embodiment of a device 10 for obtaining vital sign information of a subject 12 according to the present invention. The subject 12 lies in a bed 14, wherein the head of the subject 12 is located on a pillow 16 and the subject is covered with a blanket 18. The device 1 comprises a first detection unit 2a for acquiring a first set of detection data 3a allowing the extraction of a first vital sign information signal 4a (VS1) related to a first vital sign of the subject 12. The device 10 further comprises a second detection unit 2b for acquiring a second set of detection data 3b allowing the extraction of a second vital sign information signal 4b (VS2) related to a second vital sign of the subject 12.

The second detection unit 2b is located in the bed 14, wherein the subject 12 is lying on the second detection unit 2b and the first detection unit 2a is in a remote position relative to the subject 12. It is to be understood, that the second detection unit 2b can also be integrated into a textile structure such as the blanket 18 or the pillow 16 or can be integrated into the textiles worn by the subject 12.

The device 1 further comprises an analysis unit 5 for extracting the first vital sign information signal 4a from the first set of detection data 3a and for extracting the second vital sign information signal 4b from the second set of detection data 3b.

The device 10 further comprises a processing unit 6 for combining the first vital sign information signal 4a and the second vital sign information signal 4b to obtain a combined vital sign information signal 7 (CVS).

The device 10 further comprises an extracting unit 8 for extracting at least one of the first and second vital signs of the subject 12 from the combined vital sign information signal 7.

The analysis unit 5, the processing unit 6 and the extracting unit 8 can be implemented by separate elements (e.g. processors or software functions), but can also be represented and implemented by a common processing apparatus. Detailed embodiments the proposed device will be explained below.

FIG. 1b shows a further exemplary embodiment of a device 10a for obtaining respiratory information of the subject 12 according to the present invention. The device 10a comprises an imaging unit 20 for acquiring a first set of image data 22 detected from a skin portion 24 of the subject and for detecting a second set of image data 26 detected from a body portion 28 of the subject 12. In this first embodiment, the skin portion 24 is the forehead of the subject 12 and the body portion 28 is the chest of the subject 12. It is to be understood, that in further embodiments, the skin portion 24 can also be the arm or other detectable skin areas of the subject and the body portion can also include the mouth and/or the nose of the subject 12.

The device 10a further comprises an analysis unit 30 adapted to extract a heart rate signal 32 (compare FIG. 2a), related to a heart rate information, from the first set of image data 22. The analysis unit is further adapted to extract a motion signal 34 from the second set of image data 26, wherein the motion signal 34 is related to respiratory information of the subject 12. In this embodiment and in the following embodiments, the respiratory information is the respiratory rate or derivatives thereof and the heart rate information is the heart rate and/or derivatives thereof.

The device 10a further comprises a processing unit 36 adapted to at least partially removing the heart rate information from the motion signal 34 by use of the extracted heart rate signal 32 extracted from the first set of image data 22. The device 10a further comprises an extracting unit 38 for extracting respiratory information of the subject 12 from the motion signal 34 being processed by the processing unit 36.

The analysis unit 30, the processing unit 36 and the extracting unit 38 can be implemented by separate elements (e.g. processors or software functions), but can also be represented and implemented by a common processing apparatus.

In this setup, the imaging unit 20 is installed at a remote distance, for example at a ceiling or a wall of a room in which the bed 14 is located. A light source 40 can be present to illuminate the scenery and to ensure sufficient image contrast. In one embodiment, the imaging unit 20 can be an infrared camera and the light source 40 can be an infrared light source. It is to be understood, that in further embodiments the camera can be adapted to detect light in the visible or infrared spectral range and the light source can be adapted to emit light in the infrared and/or visible spectral range. In this embodiment, the subject 12 and the imaging unit 20 are located oppositely to one another. It is to be understood that the imaging unit 20 and/or the camera can in principle be arbitrarily oriented with respect to the subject 12.

FIG. 2a shows a sectional view of the subject's 12 forehead as an inset, wherein the imaging unit 20 detects the first set of image data 22 from the skin portion 24. The extracted heart rate signal 32 extracted by the analysis unit 30 is depicted as graph in FIG. 2a. In FIG. 2b the body portion 28 of the subject 12 is depicted as an inset. In the graphs shown in FIG. 2b the motion signal 34 extracted by the analysis unit 30 from the second set of image data 26 is shown.

The motion signal 34 is divided into a first section 42 and a second section 44. In the first section 42 the typical signal related to respiratory movement of the subject 12 is given. In a first time interval, which is defined by the first section 42 the person breaths regularly. In the second section 44 the subject 12 observed has an apnoea phase. However, a motion signal portion can be observed. This motion signal portion is related to heart rate information, or in other words a heart rate artifact 45, a so-called cardiac seismogram. It is to be understood, that the cardiac seismogram and the portion of the motion signal 34 related to respiration of the subject 12 needs not be separated that clearly in time. It is typical that both signals are superimposed over a certain time interval.

The processing unit 36 uses the heart rate signal 32 depicted in FIG. 2a for removing the superimposed heart rate artifact 45 in the second section 44 in the motion signal 34 shown in FIG. 2b in the upper part of the graph. To obtain a cleaned motion signal 48 the processing unit 36 comprises a notch filter 46 that is adapted to the heart rate signal 32 and cuts out the portion related to the heart rate artifact 45. The cleaned motion signal or in other words the cleaned respiratory signal 48 related to the respiratory information is also shown in FIG. 2b in the lower part of the graph. It can be clearly taken from the second section 44 of the cleaned motion signal 48 that during the apnea phase of the person no respiratory related motion is detected. It is to be understood, that the notch filter 46 can be used in the time and/or in the frequency domain for removing the heart rate artifact 45 being depicted in section 44 of the motion signal 34 in the first row of the graph. Several parameters being extracted from the heart rate signal 32 can be used to identify the superimposed heart rate artifact 45, e.g. the heart rate, the shape of the heart rate signal 32 and/or derivatives thereof can be taken into account. For instance, the frequency of the heart rate signal 32 or the time interval between two heart beat amplitudes (compare FIG. 2a) can be taken into account to identify the heart rate artifact 45 and to remove the same from the motion signal 34.

The second section 44 can further be used for defining a filter window 47 for the notch filter 46 allowing for distinctly separating and/or removing the heart rate artifact 45 from the motion signal 34 related to the respiration of the subject 12. In this embodiment, the filter window 47 can be adjusted by choosing a proper time interval and/or amplitude height. When using the notch filter 46 in the frequency domain the filter window 47 can be chosen such that it corresponds to the heart rate frequency or that the filter window 47 can be set within a predefined tolerance interval. In addition, the filter window 47 can further be adapted to the amplitude height of the heart rate signal 32.

In the graphs shown in FIG. 2c the motion signal 34 and the cleaned motion signal 48 shown in the first section 42 of FIG. 2b are shown in more detail. From the enlarged view of the first section 42 it can be taken, that the heart rate artifact 45 is also present during respiration of the subject 12. The heart rate artifact 45 is superimposed on the motion signal 34. After cleaning the motion signal 34 from the heart rate artifact 45 according to the methods described above, the cleaned motion signal 48 remains.

FIGS. 3 and 4 illustrate a further embodiment of a device 10b for obtaining respiratory information of the subject 12 according to the present invention. The embodiment shown in FIG. 4 is substantially based on the embodiment shown in FIG. 1. The heart rate signal 32a shown in FIG. 3a additionally comprises a superimposed further respiratory signal 48a related to the respiratory rate of the subject 12. This further respiratory signal 48a is depicted as a dashed line in the graph, which is the so-called baseline DC modulation. This modulation is related to a variation in venous pressure being modulated by the respiration of the subject 12. The heart rate signal 32a can be separated into a cleaned heart rate signal 32b shown in the upper part of FIG. 3b, and into the further respiratory signal 48a shown in the lower part of FIG. 3b. In the graph shown in the third row, the cleaned respiratory signal 48 detected from the body portion 28 of the subject is depicted.

The separation of the heart rate signal 32a into the cleaned heart rate signal 32b and the further respiratory signal 48a is performed by the analysis unit 30a according to the further embodiment of the present invention shown in FIG. 4. Therefore, the analysis unit 30a is adapted to perform continuous wavelet transformation of the heart rate signal 32a taking into account the further respiratory signal 48a as respiratory information. It is to be understood that the depicted baseline modulation is only one possible type of respiratory information, which can be taken into account as respiratory rate indicator. Further respiratory information such as pulse, amplitude modulation (PSA) and/or respiratory sinus arrhythmia (RSA) can also be taken into account to separate the heart rate signal 32a into the cleaned heart rate signal 32b and the respiratory signal 48a. To compare the first respiratory signal 48 extracted from the second set of image data 26, being detected from the body portion 28 and the further respiratory signal 48a extracted from the first set of image data 22, being detected from the skin portion 24 of the subject 12, a comparing unit 52 is provided. The comparing unit 52 compares the first respiratory signal 48 and the further respiratory signal 48a and is adapted to report the results to another remote device (not shown). Additionally, an alarm function can be provided to give alarm if the first respiratory signal 48 and the second respiratory signal 48a comprise a significant difference to one another, wherein the significant difference is derived from a predefined parameter related e.g. to the subject 12 or to conditions predefined by a user of the device 10b.

FIG. 5 shows yet a further embodiment of a device 10c for obtaining respiratory information of a subject 12 according to the present invention. The device 10c further comprises a user interface 54 for entering information allowing for selecting and/or predefining the skin portion 24 and/or the body portion 28 of the subject 12. The imaging unit 20 comprises a certain field of view 56 being sketched by the dashed lines. The field of view 56 can be chosen by a user such that at least the skin portion 24 and the body portion 28 can be detected by the imaging unit 20.

In further embodiments the imaging unit 20 can also be adapted such that the field of view can be concentrated on a smaller field of view, for example only covering the mouth and chest portions of the subject 12 lying in the bed 14. This can for example be done by a zoom objective attached to the imaging unit 20. In further embodiments, the imaging unit 20 can also be controlled by a motor to adapt the field of view for example to the chest portion of the subject 12 or to the forehead or to the arm and/or to other regions of the body being of potential interest. The device 10c according to the present embodiment additionally comprises an initializing unit 58 for selecting and/or predefining the skin portion 24 and the body portion 28 based on entered information and/or information related to the subject 12. The initializing unit 58 is adapted to use information entered via the user interface 54 and/or to use information related to the subject 12 itself. For selecting and/or predefining or even locating the skin portion 24 and the body portion 28 markers 60a, 60b are provided to select a certain region of interest. These markers may also provide a pointer to predefine the skin portion 24 and/or the body portion 28.

FIG. 6 shows another embodiment of a device 10d for obtaining vital sign information of the subject 12 according to the present invention. The device 10d comprises an imaging unit 20d for acquiring a set of image data 22d detected from the skin portion 24 of the subject 12 and from the body portion 28 of the subject 12. In this embodiment, the skin portion 24 is the forehead of the subject 12 and the body portion 28 is the chest of the subject 12. It is to be understood that in further embodiments the skin portion 24 can also be the arm or other detectable skin areas of the subject and the body portion can also include the mouth and/or the nose of the subject 12.

The device 10d further comprises a second detection unit 62 comprising capacitive sensors 64a, 64b, 64c, 64d and pressure sensors 66a, 66b, 66c, 66d. The capacitive sensors 64a, 64b, 64c, 64d and the pressure sensors 66a, 66b, 66c, 66d are located in the bed 14 and are configured to detect signals related to vital sign information, especially the heart rate and/or the respiratory rate of the subject 12. It is to be understood, that the capacitive sensors 64a, 64b, 64c, 64d and/or the pressure sensors 66a, 66b, 66c, 66d can also be integrated into a textile structure such as the blanket 18 or the pillow 16 or can be integrated into the textiles worn by the subject 12. The respiratory rate or related vital signs information are derived from the absolute pressure or pressure variations detected from the pressure sensors 66a, 66b, 66c, 66d caused by the subject 12. The heart rate or related vital sign information are detected by a variation in the local electric field caused by the heart activity of the subject 12 as known from capacitive ECG measurement. Sensor data 68a are received from the capacitive sensors 64a, 64b, 64c, 64d and further sensor data 68b are received from the pressure sensors 66a, 66b, 66c, 66d, wherein the sensor data 68a, 68b are transferred to an analysis unit 30d. It is to be understood, that the sensor data 68a and 68b represent the second set of detection data 3b as described in FIG. 1a.

The analysis unit 30d is adapted to extract the first vital sign information signal 4a from the set of image data 22d and to extract the second vital sign information signal 4b from the sensor data 68a. The first vital sign information signal 4a and the second vital sign information signal 4b are redundant and are related to the heart rate of the subject 12. It is to be understood that also other vital sign information signals related to other vital signs such as the respiratory rate, can be taken into account as long as the first vital sign information signal 4a and the second vital sign information signal 4b are related to the same vital sign.

The device 10d further comprises a processing unit 36d for combining the first vital sign information signal 4a received from the imaging unit 20d and the second vital sign information signal 4b received from the second detection unit 62 to obtain the combined vital sign information signal 7 (compare FIG. 1a). The processing unit 36d is configured to weight the first vital sign information signal 4a by use of a first quality index for receiving a weighted first vital sign information signal 70 and to weight the second vital sign information signal 4b by use of a second quality index to receive a weighted second vital sign information signal 72 and to combine the weighted first vital sign information signal 70 (WVS1) and the weighted second vital sign information signal 72 (WVS2) to obtain a weighted vital sign information signal 74, representing the combined vital sign information signal 7 (compare FIG. 1a).

The quality indices used for weighting the first vital sign information signal 4a and the second vital sign information signal 4b are derived from the set of image data 22d and from the second detection data 3b received from the second detection unit 62. In particular, the first and second quality indices are derived from one or more features of the set of image data and/or environmental data of the environment of the subject 12, comprising one or more of an illumination parameter of the subject's illumination, an amplitude, shape and/or variability shape of the first vital sign information signal 4a and motion artifacts detected by the detection unit 62. Moreover, the signal to noise ration, the variability or the shape of the specific vital sign information signals can further be taken into account. In addition, the resistance of the electrodes used can further be taken into account.

The device 10d further comprises an extracting unit 38d for extracting at least one of the first and second vital signs of the subject 12 from the weighted vital sign information signal 74.

The analysis unit 30d, the processing unit 36d and the extracting unit 38d can be implemented by separate elements (e.g. processors or software functions), but can also be represented and implemented by a common processing apparatus.

In this setup, the imaging unit 20d is installed at a remote distance for example at a ceiling or a wall of a room in which the bed 14 is located. The light source 40 can be present to illuminate the scenery and to ensure sufficient image contrast. In one embodiment, the imaging unit 20 can be an infrared camera and the light source 40 can be an infrared light source. It is to be understood, that in further embodiments the camera can be adapted to detect light in the visible or infrared spectral range and the light source can be adapted to emit in the infrared and/or visible spectral range. In this embodiment, the subject 12 and the imaging unit 20d are located opposite to one another. It is to be understood that the imaging unit 20d can in principle be arbitrarily oriented with respect to the subject 12. The weighted vital sign information signal 74 is received by combining the first weighted vital sign information signal 70 and the second weighted vital sign information signal 72 using a weighting scheme, e.g. the arithmetic or geometric mean or by simply picking the vital sign information signal having a maximum quality index.

FIG. 7 shows a process flow for obtaining vital sign information of a subject. In the first step S1 a first set of detection data is acquired, allowing the extraction of a first vital sign information signal related to a first vital sign of the subject. In a second step S2 a second set of detection data is acquired, allowing the extraction of a second vital sign information signal related to a second vital sign of the subject. The first set of detection data contains vital sign information, from which a first vital sign information signal is extracted from in step S3. The second set of detection data contains second vital sign information, from which the second vital sign information signal is extracted from in step S4. The first vital sign information signal and the second vital sign information signal are combined to obtain a combined vital sign information signal in step S5. At least one of the first and second vital signs is extracted from the combined vital sign information signal S6.

Conventional camera-based respiration monitoring is realized by measuring the subtle breathing motion in the subject's chest (or belly) area. So it critically depends on the detection of subtle breathing motion in the video. The motion-based respiratory signal monitoring is not always reliable, due to the difficulty of breathing motion detection in certain cases. For example, a neonate in the NICU sometimes has shallow breathing, where it is challenging to detect the very subtle breathing motion. If the algorithm parameters are adjusted to be sensitive enough to the very subtle motion of shallow breathing, another problem will arise: the algorithm cannot differentiate the subtle breathing motion from the noises (illumination, camera, etc.). For instance, pointing to the wall, the algorithm can produce the respiration-like signal due to the noises.

Another way to derive the respiratory signal remotely is by processing the photoplethysmography (PPG) signals calculated from the video. It is generally known in the art that a respiratory signal can be extracted from PPG signals, and that PPGs signal can be derived remotely by measuring the change in the skin area (called remote PPG or R-PPG). Recent experiments of the inventors have shown it is possible to extract the respiratory signal from the remote PPG signal derived from the vital signs camera. However, R-PPG-based respiration monitoring also has limitations; for example, PPG signal could be sensitive to subject's motion, ambient illumination (changes), camera noises, etc.

FIG. 8 shows a further embodiment of the device 10e for obtaining respiratory information of the subject according to the present invention in a more accurate and reliable way. The device 10e is similar to the device 10b shown in FIG. 4 and like elements are numbered with like reference numerals.

The device 10e particularly comprises an imaging unit 20, in particular a camera representing the first detection unit and the second detection unit, for acquiring a first set of image data 22 detected from a skin portion 24 of the subject 12 allowing the extraction of a first respiratory signal 80 related to a respiratory information of the subject 12 and a second set of image data 26 detected from a body portion 28 of the subject 12 allowing the extraction of a second respiratory signal 82 related to respiratory information 48 of the subject 12. The first and second respiratory signals 80, 82 are extracted by the analysis unit 30e. The processing unit 36e is configured to weight the first respiratory signal 80 by use of a first quality index, to weight the second respiratory signal 82 by use of a second quality index and to combine the weighted respiratory signal 84 and the weighted motion signal 86 to obtain a weighted combined respiratory signal 88. By the extraction unit 38 the final respiratory signal 90, e.g. the respiratory rate, of the patient is obtained.

Thus, according to this embodiment reliable camera-based respiration monitoring is provided by combining the respiratory signal extracted from breathing motion and the respiratory signal extracted from the PPG signal. One or more cameras are used to monitor the subject. The acquired video contains at least one part of the body showing breathing motion (e.g., the chest and/or belly) and at least one part of skin area. The acquired video is analysed to derive the respiratory signal in two ways: one is based on breathing motion detection, while the other is from the PPG signal. A quality index could be calculated for individual respiratory signal at the same time. The two types of respiratory signals are combined, based on the quality indexes or not, to derive the output respiratory signal (and quality index). It is also possible to further combine with the respiratory signals from multiple regions of interest, from different cameras, or from other (contact or contactless) sensors.

For camera-based respiration monitoring, breathing motion based measurement and PPG based measurement can complement each other to improve reliability and robustness, since they have different strength and limitations in certain cases. For example, when the neonate has shallow breathing, the motion-based measurement could be less reliable, but the PPG-based measurement is more reliable. On the other hand, if there are ambient illumination changes (or shallow effects), it could be noisy to extract the PPG signal, but the motion-based measurement can be more reliable.

It is possible to monitor the subject with one camera (as shown in FIG. 8). The acquired video contains at least one part of the body showing breathing motion (e.g., the chest and/or belly) and at least one part of skin area. In practice, multiple cameras may be used, for example, one camera can zoom in to the skin area to extract PPG, while the other camera looks at the patient's chest/belly to measure breathing motion. The acquired video is analysed to derive the respiratory signal based on the breathing motion and from the PPG signal. A quality index could be calculated for each respiratory signal at the same time. The quality index can be calculated based on the respiratory signal itself, or information extracted from the video or other context information, for example, signal to noise ratio, shape of respiratory signal versus expected physiological pattern, motion artefact, and so on.

The two respiratory signals are combined, based on the quality indexes or not, to derive the output respiratory signal (and an overall quality index). The combination can be done by various methods such as logic (e.g., the one with the best quality is used) and a weighting scheme. A threshold for quality index can be defined. The respiratory signal with the quality index below the threshold will not be considered “accepted”. In one embodiment, a simple combination method is to select the signal with the better quality. If both signals have the quality index below the threshold, there will be no output. In another embodiment, if both signals have the quality index above the threshold, instead of selecting the better one, the final output could be a fusion of two signals, for example, a weighted average of both signals, where the weight factor depends on the quality index.

Furthermore, multiple ROIs (for motion-based measurement or PPG-based measurement) in a single video stream or a plurality of different cameras can each be regarded as an input for the signal combination.

It is also possible to further combine with the respiratory signal from other contact or contactless sensors, for example, pressure sensor based measurement.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or an does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

1. A device for obtaining vital sign information of a subject, comprising:
a first detection unit that acquires a first set of detection data allowing the extraction of a first vital sign information signal related to a first vital sign of the subject,
a second detection unit that acquires a second set of detection data allowing the extraction of a second vital sign information signal related to a second vital sign of the subject,
an analysis unit that extracts the first vital sign information signal from the first set of detection data and that extracts the second vital sign information signal from the second set of detection data,
a processing unit that combines the first vital sign information signal and the second vital sign information signal to obtain a combined vital sign information signal, and
an extracting unit that extracts at least one of the first and second vital signs of the subject from the combined vital sign information signal.

2. The device as claimed in claim 1, comprising:
an imaging unit, representing the first detection unit and the second detection unit, that acquires a first set of image data detected from a skin portion of the subject allowing the extraction of a heart rate signal related to a heart rate of the subject and a second set of image data detected from a body portion of the subject allowing the extraction of a motion signal related to respiratory information of the subject,
wherein the analysis unit is configured to extract the heart rate signal from the first set of image data and the motion signal from the second set of image data, wherein the motion signal comprises a superposition of respiratory information and heart rate information, wherein the processing unit is configured to at least partially remove the heart rate information from the motion signal by use of the extracted heart rate signal, and
wherein the extracting unit is configured to extract respiratory information of the subject from the processed motion signal.

3. The device as claimed in claim 2, wherein the imaging unit comprises a single camera that detects electromagnetic radiation at least in the visible and/or infrared spectral range.

4. The device as claimed in claim 2, wherein the imaging unit is configured to simultaneously acquire the first set of image data and the second set of image data.

5. The device as claimed in claim 2, wherein the processing unit comprises a notch filter triggered by the heart rate signal and allowing for at least partially removing the heart rate information from the motion signal.

6. The device as claimed in claim 5, wherein the notch filter comprises filter windows that are dynamically adaptable to the heart rate signal.

7. The device as claimed in claim 2, wherein the heart rate signal comprises superimposed further respiratory information, wherein the analysis unit is configured to extract further respiratory information by use of the heart rate signal.

8. The device as claimed in claim 7, wherein the analysis unit is adapted to perform continuous wavelet transformations of the heart rate signal allowing for extracting further respiratory information from the heart rate signal.

9. The device as claimed in claim 7, further comprising a comparing unit that compares respiratory information extracted from the motion signal and further respiratory information extracted from the heart rate signal.

10. The device as claimed in claim 2, further comprising a user interface that enters information allowing for selecting and/or predefining the skin portion and the body portion.

11. The device as claimed in claim 10, further comprising an initializing unit that selects and/or predefines the skin portion and the body portion based on entered information and/or information related to the subject.

12. The device as claimed in claim 1,
wherein the first detection unit comprises an imaging unit that acquires a set of image data representing the first set of detection data allowing the extraction of the first vital sign information signal related to the first vital sign of the subject,
wherein the second detection unit comprises a sensor unit that acquires a second set of sensor data detected from a body portion of the subject representing the second set of detection data allowing the extraction of the second vital sign information signal related to the first vital sign of the subject, wherein the first vital sign and the second vital sign are identical,
wherein the processing unit is configured to weight the first vital sign information signal by use of a first quality index, to weight the second vital sign information signal by use of a second quality index and to combine the weighted first vital sign information signal and the weighted second vital sign information signal to obtain the weighted vital sign information signal.

13. The device as claimed in claim 12, wherein the processing unit is configured to derive the first quality index from the set of image data and the second quality index from the set of sensor data.

14. The device as claimed in claim 12, wherein the processing unit is configured
to derive the first quality index from one or more features of the set of image data and/or environmental data of the environment of the subject, in particular from one or more of an illumination parameters of the subject's illumination, an amplitude, shape and/or variability shape of the first vital sign information signal and motion artifacts, and
to derive the second quality index from the set of sensor data from one or more features of sensor data and/or environmental data of the environment of the subject.

15. The device as claimed in claim 12, wherein the sensor unit comprises one or more capacitive sensors that acquire ECG information of the subject and/or pressure sensors that acquire weight information of the subject.

16. The device as claimed in claim 1, comprising:
an imaging unit, representing the first detection unit and the second detection unit, for acquiring a first set of image data detected from a skin portion of the subject allowing the extraction of a first respiratory signal related to a respiratory information of the subject and a second set of image data detected from a body portion of the subject allowing the extraction of a second respiratory signal related to respiratory information of the subject, wherein the processing unit is configured to weight the first respiratory signal by use of a first quality index, to weight the second respiratory signal by use of a second quality index and to combine the weighted respiratory signal and the weighted motion signal to obtain a weighted combined respiratory signal.

17. A method for obtaining vital sign information of a subject, comprising the steps of:
acquiring a first set of detection data allowing the extraction of a first vital sign information signal related to a first vital sign of the subject,
acquiring a second set of detection data allowing the extraction of a second vital sign information signal related to a second vital sign of the subject,
extracting the first vital sign information signal from the first set of detection data and extracting the second vital sign information signal from the second set of detection data,
combining the first vital sign information signal and the second vital sign information signal to obtain a combined vital sign information signal, and
extracting at least one of the first and second vital signs of the subject from the combined vital sign information signal.

18. A processing apparatus for obtaining vital sign information of a subject, comprising:
an analysis unit that extracts a first vital sign information signal from a first set of detection data and that extracts a second vital sign information signal from a second set of detection data,
a processing unit that combines the first vital sign information signal and the second vital sign information signal to obtain a combined vital sign information signal, and
an extracting unit that extracts at least one of the first and second vital signs of the subject from the combined vital sign information signal.

19. A processing method for obtaining vital sign information of a subject, comprising the steps of:
extracting a first vital sign information signal from a first set of detection data and extracting a second vital sign information signal from a second set of detection data,
combining the first vital sign information signal and the second vital sign information signal to obtain a combined vital sign information signal, and
extracting at least one of the first and second vital signs of the subject from the combined vital sign information signal.

20. A computer readable non-transitory medium having instructions stored thereon which, when carried out on a computer, cause the computer to perform the steps of the method as claimed in claim 17.
